(19) United States
(12) Patent Application Publication
(10) Pub. No.: US 2018/0299447 A1
(43) Pub. Date: Oct. 18, 2018

(54) CERVICAL CANCER TESTING METHOD AND TEST REAGENT USED THEREFOR

(71) Applicant: TOKYO MEDICAL UNIVERSITY, Tokyo (JP)

(72) Inventors: Masahiko KURODA, Tokyo (JP); Koji FUJITA, Tokyo (JP); Shin-ichiro OHNO, Tokyo (JP); Atsushi KURATA, Tokyo (JP); Yuichirou HARADA, Tokyo (JP); Hirotaka NISHI, Tokyo (JP)

(21) Appl. No.: 15/766,630

(22) PCT Filed: Jun. 30, 2016
(86) PCT No.: PCT/JP2016/069468

§ 371 Date: Apr. 6, 2018

(30) Foreign Application Priority Data
Oct. 8, 2015 (JP) 2015-200598

(51) Int. Cl. G01N 33/574; C07K 16/30

(52) U.S. Cl. CPC G01N 33/57411; C07K 16/3092

(57) ABSTRACT

The present invention aims to provide a novel test method for evaluating the possibility of cervical cancer and a novel test reagent for use in the method. The present invention provides a test method for evaluating the possibility of cervical cancer, including the step of: detecting the MUC expression in a biological sample isolated from the uterine cervix.

### TECHNICAL FIELD

The present invention relates to a test method for cervical cancer and a test reagent for use in the method.

### BACKGROUND ART

The cervical cancer screening based on cytology is one of a few screening tests that have been proved to contribute to a reduction in mortality rate. A general procedure of the cervical cancer screening is as follows. First, using a specimen scraped from the uterine cervix, a Papanicolaou (Pap.)-stained specimen slide is prepared. The slide is examined microscopically by a cytotechnologist and a pathologist to detect the presence of human papilloma virus (HPV)-infected epithelial cells having characteristic morphology. The risk of cervical cancer is determined by classifying the progression etc. of precancerous lesions according to the Bethesda system. Early detection of the precancerous lesions by this method is considered to be helpful in treatment of cervical cancer patients.

In recent years, in order to deal with the earlier age of onset of cervical cancer, it was determined that the cervical cancer screening should be taken every two years beginning at age 20. While the cervical cancer screening uptake rate is still low (at the 20% level), the number of women taking the cervical cancer screening has increased by a factor of 2.5 in the last ten years. It is expected that the demand for cervical cancer screening will increase still further, especially in consideration of the fact that the major cause of cervical cancer is HPV infection transmitted sexually and the recent trend toward the start of sexual activities at an earlier age, for example. However, in the cervical cancer screening based on cytology, the determination is made manually by specialists including a cytotechnologist. As a matter of course, the cervical cancer screening requires much labor and cost, and the cost required for the cervical cancer screening places considerable burden on municipalities. Further, the morphology of epithelial cells used in the cytology varies depending on the age of a subject, the presence or absence of inflammation, and the hormonal environment. Accordingly, in some cases, it might be difficult to make the determination. Moreover, since the determination is made manually, it is necessary that the cytotechnologist and the pathologist have sufficient skill and experience. Thus, the determination may vary depending on the cytotechnologist and the pathologist.

Under these circumstances, attempts have been newly made to use the following method or test in combination with the cytology: a method for detecting HPV-derived proteins (Patent Document 1) or an HPV test for checking HPV infection by detecting mRNA or DNA of HPV (Patent Document 2). However, in the HPV test, it is necessary to extract a gene from biological tissue and then amplify the gene. Thus, introduction of the HPV test poses a problem of cost, manpower, etc. Moreover, it has been pointed out that the gene amplification might cause a problem of false positive or false negative results owing to the specificity of a primer set, for example.

### CITATION LIST

### Patent Document(s)

Patent Document 1: Japanese Translation of PCT International Application Publication No. JP-T-2013-516623

Patent Document 2: Japanese Translation of PCT International Application Publication No. JP-T-2008-528058

### BRIEF SUMMARY OF THE INVENTION

### Problem to be Solved by the Invention

With the foregoing in mind, it is an object of the present invention to provide a novel test method for evaluating the possibility of cervical cancer and a novel test reagent for use in the method.

### Means for Solving Problem

In order to achieve the above object, the present invention provides a test method for evaluating a possibility of cervical cancer, including the step of: detecting expression of a MUC in a biological sample isolated from the uterine cervix.

The present invention also provides a test reagent for cervical cancer, containing: a binding substance that binds to a MUC.

### Effects of the Invention

According to the present invention, it is possible to determine the possibility of cervical cancer easily by merely detecting the presence or absence of a MUC in a biological sample isolated from the uterine cervix.

### BRIEF DESCRIPTION OF DRAWINGS

FIGS. 1A to 1D show immunostaining results indicating the MUC1 expression in specimens from CIN patients in Example 1.

FIGS. 2A to 2C show immunostaining results indicating the MUC1 expression in LBC specimens from cervical cancer patients in Example 2.

FIGS. 3A and 3B show immunostaining results indicating the MUC1 expression in specimens from a CIN patient and a SCC-positive patient in Example 3.

FIGS. 4A to 4D show immunostaining results indicating the MUC1 expression in specimens from CIN patients and a SCC-positive patient in Example 3.

FIGS. 5A and 5B show immunostaining results indicating the MUC1 expression in a specimen from a CIN patient in Example 4.

FIGS. 6A to 6C show immunostaining results indicating the MUC1 expression in endometrial glandular cells in Example 5.

FIGS. 7A to 7D show immunostaining results indicating the MUC1 expression in specimens from cervical cancer patients in Example 6.

FIGS. 8A to 8D show immunostaining results indicating the MUC1 expression in specimens from other cervical cancer patients in Example 6.

FIGS. 9A to 9D show immunostaining results indicating the MUC1 expression in specimens from still other cervical cancer patients in Example 6.

FIGS. 10A to 10C show immunostaining results indicating the MUC1 expression in specimens from patients determined to be NILM in Example 7.

### MODE FOR CARRYING OUT THE INVENTION

<Test Method>

The test method according to the present invention is, as described above, a test method for evaluating a possibility of cervical cancer, including the step of: detecting expression of a MUC in a biological sample isolated from the uterine cervix.

MUCs, which form mucin core proteins, are known as components of mucus. Mucus is not present in the uterine cervical epithelium. Thus, it has been common general technical knowledge that MUCs are not present in an epithelium of the uterine cervix. However, the inventors of the present invention found through diligent research that, while MUC expression is not observed in a normal epithelium, specific phenotypic expression of MUCs is observed in an epithelium of a subject with cervical intraepithelial neoplasia. On the basis of this finding, the inventors of the present invention discovered that the possibility of cervical cancer can be evaluated by detecting the presence of a MUC in a biological sample isolated from an epithelium of the uterine cervix. The MUC expression is not observed in a normal epithelium. Thus, according to the present invention, it is possible to determine whether a subject has cervical cancer depending on the presence or absence of a MUC, for example. Also, since the MUC expression is not observed in a normal epithelium, the present invention can avoid the problem of false positive or false negative results, so that highly reliable results can be obtained. Moreover, the present invention only requires checking the presence or absence of a MUC. The detection of the MUC can be performed by selecting an existing technique for detecting a target substance as appropriate. Accordingly, the present invention can realize cost reduction, simplification, automation, and the like of the cervical cancer screening, for example.

In the above-described cytology, the specimens are classified into the types listed in Table 1 below according to the Bethesda system. Under the current circumstances, 90% to 95% of the specimens are determined to be negative for intraepithelial lesion or malignancy (NILM), which falls under the negative result in the Bethesda system. In contrast, according to the test method of the present invention, first, specimens accounting for 90% to 95% of all the specimens can be determined to be negative on the basis of the fact that a MUC is not detected by the test method of the present invention, for example. Therefore, the above-described cytology further may be conducted only for the specimens in which the MUC is detected by the test method of the present invention. This can drastically reduce the cost and labor for the cervical cancer screening.

TABLE 1

| | |
|---|---|
| NILM | negative for intraepithelial lesion or malignancy |
| ASC-US | atypical squamous cells of undetermined significance |
| ASC-H | atypical squamous cells that cannot exclude HSIL |
| LSIL | low grade SIL |
| HSIL | high grade SIL |
| SCG | squamous cell carcinoma |

As described above, the present invention is characterized in that it detects the MUC expression in the biological sample, and other conditions, steps, etc. are by no means limited. In the detection step, the detection of the MUC expression may be, for example, detection of the presence or absence of the MUC (qualitative analysis) or detection of the amount of the MUC (quantitative analysis).

The test method according to the present invention excludes acts by physicians, for example.

In the test method according to the present invention, it is determined that the biological sample has a possibility of cervical cancer when the MUC is detected in the biological sample in the detection step, for example.

In the present invention, the detection step may be a step of detecting the expression of the MUC using a binding substance that binds to the MUC, for example. When the binding substance is used, the presence or absence of binding of the binding substance with the MUC correlates with the presence or absence of the MUC, and the amount of binding of the binding substance with the MUC correlates with the amount of the MUC, for example. Accordingly, the expression of the MUC can be detected indirectly by detecting the binding. For example, descriptions regarding the test reagent of the present invention to be provided below also apply to the binding substance in the present invention, and the term “binding substance” is used interchangeably with the test reagent of the present invention.

In the present invention, in the detection step, the expression of the MUC can be detected indirectly by detecting the binding substance that has bound to the MUC, for example.

In the present invention, in the detection step, the expression of the MUC is detected by detecting a signal generated directly or indirectly through binding of the binding substance with the MUC, for example. The signal can be generated by labeling the binding substance with a labeling substance to be described below, for example.

The binding substance is not particularly limited as long as it binds to a MUC. Preferably, the binding substance is a substance that specifically binds to the MUC, for example. It is also preferable that the binding substance binds to the MUC and substantially does not bind to any substances other than the MUC contained in the biological sample isolated from the uterine cervix, for example.

Examples of the binding substance include: proteins such as antibodies; peptides such as antigen-binding fragments of antibodies; and nucleic acids. Among them, antibodies, i.e., anti-MUC antibodies, are preferable from the viewpoint of ease of detecting the binding with a target.

The binding between the binding substance and the MUC can be detected using a conventionally known method for detecting the binding between a target and a binding substance that binds to the target, for example. When the binding substance is an antibody, a method for detecting an antigen-antibody reaction can be used, for example. Specific examples of the method include enzyme-linked immunosorbent assay (ELISA), radioimmunoassay (RIA), immunochromatography assay, immunostaining such as immunohistochemical staining, and flow cytometry. The binding substance may be labeled with an appropriate substance depending on the type of the detection method, and examples of the substance include: labeling substances such as enzymes, radioisotopes, and particles; dyes such as fluorescent dyes; and color developing substrates for enzymes. The particles may be particles of a metal such as gold or silver or latex particles such as colored latex particles, for example. The binding substance may be used in combination with a reducing agent such as a substrate for an enzyme or divalent iron (Fe²⁻) depending on the type of the detection method, for example.

When the binding substance is an antibody, a primary antibody that binds to the MUC (anti-MUC antibody) may be used alone, or alternatively, a primary antibody that binds to the MUC (anti-MUC antibody) and a secondary antibody that binds to the primary antibody may be used in combination, for example. In the former case, the detection of the MUC can be achieved by detecting the binding of the primary antibody with the MUC, for example. In the latter case, the detection of the MUC can be achieved by detecting the binding of the secondary antibody to the primary antibody that has bound to the MUC, for example. In the former case, the primary antibody preferably is a labeled antibody labeled with the above-described labeling substance, for example. In the latter case, the secondary antibody preferably is a labeled antibody labeled with the above-described labeling substance, for example.

MUCs belong to a family of mucin core proteins as described above, and examples thereof include MUC1, MUC2, MUC3, MUC4, MUC5AC, MUC5B, MUC6, and MUC7. In the present invention, one type of MUC or two or more types of MUCs may be detected, for example. The MUC to be detected preferably is MUC1, for example. Examples of a target other than MUCs include molecules expressed in the course of differentiation from a squamous epithelium to glandular tissue, Cytokeratin 5/6, Cytokeratin 7, Cytokeratin 8, Cytokeratin 17, Cytokeratin 18, Cytokeratin 19, BCA 225, CA 15-3, CA 19-9, CA 50, CA 54/61, CA 72-4, CA 125, CA 130, CA 602, CSLEX, DUPAN-2, KMO-1, NCC-ST-439, SLX, SPan-1, STN, and CYFRA. In the present invention, the expression of any of these other targets may be detected in addition to the expression of the MUC, for example.

In the present invention, the biological sample may be cells or tissue, for example. Preferably, the biological sample is derived from an epithelium of the uterine cervix, for example. The biological sample may be in the form of solid or liquid. When the biological sample is liquid, it may be, for example, a homogenate obtained by homogenizing cells or tissue in a solvent, or a suspension obtained by suspending cells or tissue in a solvent. The type of the solvent is not particularly limited, and the solvent may be water, physiological saline, a buffer solution, a preservation solution for cells or tissue, or the like.

The biological sample may be obtained by scraping cells or tissue from the uterine cervix. Alternatively, in a sample provided for the above-described cytology, residual part not used for preparation of a specimen slide may be used as the biological sample, for example. In recent years, a liquid-based cytology (LBC) method is used widely for the preparation of a specimen slide to be used in the cytology. The LBC method is a method for preparing a specimen slide by scraping cells from the uterine cervix with a dedicated brush, washing the dedicated brush in an LBC-dedicated preservation solution to suspend the cells, and smearing the thus-obtained cell suspension uniformly on a microscope slide. This method can avoid a problem of improper specimen slides, such that correct determination cannot be made owing to an insufficient amount of the cells, the dried state of the cells, and the like, for example. On this account, in the present invention, the cell suspension may be used as the biological sample, for example. When the cell suspension is used, it is preferable to use flow cytometry in combination with the test method of the present invention, for example. The dedicated brush may be a broom-type brush (e.g., Cervex-Brush), for example.

As the dedicated preservation solution, a commercially available product may be used, for example.

<Test Reagent>

The test reagent according to the present invention is, as described above, a test reagent for cervical cancer, containing a binding substance that binds to a MUC. The test reagent of the present invention can be used in the test method according to the present invention. The test reagent of the present invention is characterized in that it contains the binding substance, and other conditions or configurations are by no means limited. Unless otherwise stated, the above descriptions regarding the binding substance in the test method of the present invention also apply to the test reagent of the present invention.

In the test reagent of the present invention, the binding substance is not particularly limited, and examples thereof are as described above. Among them, anti-MUC antibodies are preferable.

<Diagnostic Method and Diagnostic Reagent>

The present invention also provides a diagnostic method for diagnosing cervical cancer, including the step of: detecting expression of a MUC in a biological sample isolated from the uterine cervix, for example. The diagnostic method of the present invention is characterized in that it detects the MUC expression in the biological sample, and other conditions or steps are by no means limited. The above descriptions regarding the test method of the present invention also apply to the diagnostic method of the present invention. The diagnostic method of the present invention may include acts by physicians.

The present invention also provides a diagnostic reagent for diagnosing cervical cancer, containing a binding substance that binds to a MUC. The diagnostic reagent of the present invention can be used in the diagnostic method of the present invention. The diagnostic reagent of the present invention is characterized in that it contains the binding substance, and other conditions or configurations are by no means limited. Unless otherwise stated, the above descriptions regarding the test reagent of the present invention also apply to the diagnostic reagent of the present invention.

Preferably, the diagnostic method of the present invention is carried out in combination with cytology. For example, biological samples are collected from patients. Each biological sample is separated so that part thereof is used as a specimen for cytology and other part thereof is used as a specimen for MUC detection. Then, for only the biological samples determined to be MUC positive by the MUC detection, cytology is performed using the specimens for cytology. Such a method can eliminate the necessity of performing cytology for a large number of biological samples, thereby allowing the cost and labor to be reduced drastically, as described above.

### EXAMPLES

The present invention will be described specifically below with reference to examples. It is to be noted, however, that the present invention is by no means limited to these examples.

### Example 1

Biological tissues were collected from epithelia of the uterine cervices of 30 patients determined to have cervical intraepithelial neoplasia (CIN) by cytology. Regarding the thus-obtained specimens, the expression of MUC1 was examined.

In the examination of the MUC1 expression, an anti-MUC1 antibody [Ma695] (trade name: Novocastra™ Lyophilized Mouse Monoclonal Antibody Muc-1 Glycoprotein NCL-MUC-1, clone name: Ma695; cell line ZR75-1, available from Leica) or another anti-MUC1 antibody (trade name: Anti-MUC1 antibody [M4H2] ab10120, available from Abcam, clone name: M4112, epitope GVTSAPDTRPAPGSTAPPAHGVTSA) was used as a primary antibody, and a reagent composed of a dextran polymer with an anti-mouse IgG and HRP bound to (trade name: EnVision+Kit K4007, available from DAKO) was used as a secondary antibody. The MUC1 expression was examined by checking the color development of DAB, which is a substrate for HRP.

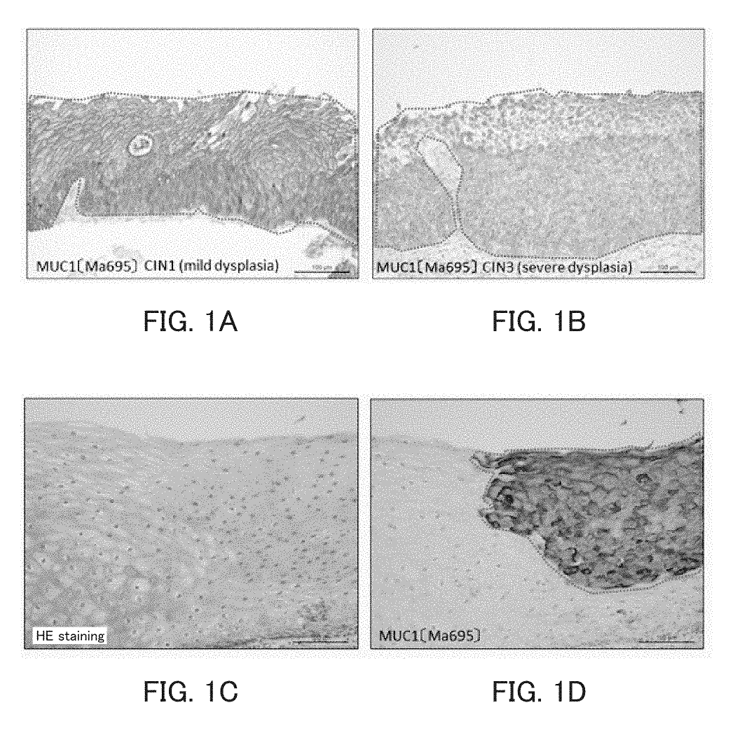

As a result, in the specimens of 27 patients, staining with the anti-MUC1 antibody was observed, from which it was found that MUC1 was expressed in these specimens. FIGS. 1A to 1D show some of the results obtained. FIGS. 1A to 1D show the results of immunostaining using the anti-MUC antibody. FIGS. 1A to 1D show, as representative examples of the results obtained regarding the 30 specimens, the results obtained regarding the specimen of a patient classified as CIN1 (mild dysplasia) and the specimen of a patient classified as CIN3 (severe dysplasia) under the CIN classification. In FIGS. 1A to 1D, the bar indicates a length of 100 μm, and the regions outlined with a dotted line are stained regions. FIG. 1A shows the result obtained regarding the CIN1 specimen. FIG. 1B shows the result obtained regarding the CIN3 specimen. FIG. 1C shows the result of HE staining of an area around the boundary between a CIN1 lateral invasive region and a normal mucosa in the C1N1 specimen. FIG. 1D shows the result of the MUC1 staining of a serial section of the specimen shown in FIG. 1C. From these results, it was found that the immunostaining using the anti-MUC1 antibody could clarify the CIN invasive region, while it was not clear in the case of the HE staining. That is, it can be said that whether a patient has CIN can be determined by detecting the MUC1 expression.

As can be seen in FIGS. 1A and 1B, in the specimens of both the CIN patients, staining with the anti-CIN antibody was observed, which revealed the ectopic expression of MUC1. In contrast, as can be seen in FIGS. 1C and 1D, MUC1 expression was not observed in the normal cervical squamous epithelial tissue.

### Example 2

Biological tissues were collected from epithelia of the uterine cervices of patients diagnosed as having cervical cancer by cytology, and specimens for liquid-based cytology (LBC) were prepared. Regarding these specimens, the expression of MUC1 was examined.

Using an LBC kit, ThinPrep® (Hologic, Inc.), specimens for LBC were prepared, and cells were smeared to provide prepared slides in the following manner.
(1) A specimen for LBC was put in a container of the kit and mixed well. Then, with a disposable pipette, 10 ml of the LBC cell suspension was collected from the container and transferred to a Spitz tube. The Spitz tube was centrifuged at 800 G for 2 minutes. Thereafter, the supernatant was removed.
(2) 1% carbowax-containing 50% ethanol (5 ml) was added to the sediment in the Spitz tube to resuspend the sediment. The Spitz tube was allowed to stand still for 5 minutes. Thereafter, the Spitz tube was centrifuged at 800 G for 2 minutes, and the supernatant was removed further.
(3) The sediment in the Spitz tube was sucked up with the disposable pipette used in the above item (1). A proper amount of the sediment (0.5 to 1 ml) was smeared onto a region positioned at one-third length from the lower edge of each of two microscope slides. Then, the smeared cells were dried completely in an incubator for 30 minutes.
(4) A staining vessel containing Japanese Pharmacopoeia ethanol was provided. The dried microscope slides were immersed in the ethanol for at least 30 minutes to immobilize the cells.
(5) After the immobilization, the dried microscope slides were immersed in a vessel containing distilled water for 5 minutes, and then transferred to an immunostaining vessel containing PBS. The microscope slides thus pretreated for staining were used for immunostaining.

Next, regarding the specimen immobilized on the microscope slides, the MUC1 expression was examined by immunostaining. In the examination of the expression, the anti-MUC1 antibody [Ma695] (trade name: Novocastra) used in Example 1 was used as a primary antibody, and one of the following three types of color-developing/fluorescence systems (a) to (c) was used as a secondary antibody.
(a) a secondary antibody labeled with Alexa Fluor 488 trade name: Alexa Fluor 488, available from Life Technologies, goat anti-mouse IgG
(b) a secondary antibody composed of a dextran polymer with an anti-mouse IgG and HRP bound thereto trade name: EnVision™+Kit, K4007, available from DAKO, peroxidase (HRP)-labeled goat anti-mouse IgG color developing substrate: DAB
(c) a secondary antibody composed of biotin-labeled anti-mouse IgG with alkaline phosphatase (AP)-labeled streptaviclin bound thereto
trade name: Histofine SAB-AP kit
color developing substrate: Fast Red
trade name: Vulcam Fast Red Chromogen Kit2, available from Biocare Medical

Specifically, the expression was examined in the following manner.
(1) The microscope slides were immersed in PBS for a short time, and the surplus PBS outside the cell smear regions was wiped away. Thereafter, the microscope slides were arranged in a humid chamber. 100 μ1 of the anti-MUC1 antibody diluted 500-fold was added dropwise onto each of the microscope slides, so that the anti-MUC1 antibody spread over the entire smear surface of the microscope slide. Then, after the reaction for 30 minutes, the microscope slides were washed three times with PBS, and the following staining step (2a), (2b) or (2c) was performed depending on the color-developing system or the fluorescence system being used.
(2a) The Alexa Fluor 488-labeled secondary antibody described in the above item (a) was diluted 200-fold, and reacted with the microscope slides for 1 hour in the humid chamber under light-shielded conditions. Thereafter, the microscope slides were washed three times with PBS. The microscope slides were subjected to nuclear staining with DAPI diluted 1000-fold with PBS for about 10 seconds, and then washed three times with PBS. The microscope slides were treated for fluorescent dye inclusion, and then observed under a fluorescence microscope.
(2b) In the case of color development with DAB, the microscope slides were reacted using the kit (EnVision™ Kit) described in the above item (c) for 30 minutes, and then washed three times with PBS. Thereafter, the microscope slides were treated with the color developing substrate DAB, and a color-developing reaction was allowed to proceed for 30 seconds. Subsequently, the microscope slides were subjected to nuclear staining with hematoxylin for about 10 seconds, and then observed under an optical microscope.
(2c) In the case of color development with Fast Red, the microscope slides were reacted with the secondary antibody described in the above item (c) for 30 minutes in a humid chamber. After the reaction, the microscope slides were washed three times with Tris-buffered saline (TBS), and subjected to a staining reaction with AP-labeled streptavidin (available from DAKO) diluted 200-fold for about 30 minutes in the humid chamber. Then, the microscope slides were washed three times with TBS, and subjected to a color-developing reaction using the kit (Vulcam Fast Red Chromogen Kit) described in the above item (c) for 10 minutes. Subsequently, the microscope slides were washed with running water, and then subjected to nuclear staining with hematoxylin. Thereafter, the microscope slides were washed again with water and then passed through distilled water. The microscope slides were treated for inclusion using a water-soluble inclusion agent, and then observed under an optical microscope.

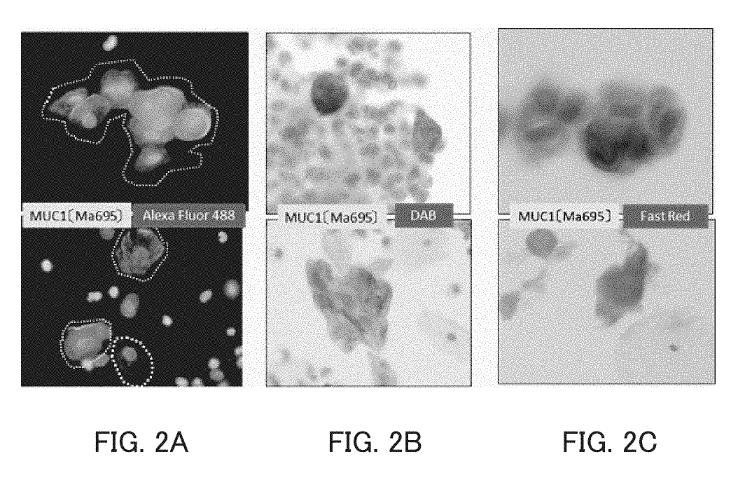

The results obtained are shown in FIGS. 2A to 2C. FIGS. 2A to 2C show the results of the immunostaining using the anti-MUC antibody. FIG. 2A shows, as representative examples of the results obtained regarding the 30 specimen, the results obtained regarding the specimen of a patient determined to have HSIL (moderate dysplasia) and the result obtained regarding the specimen of a patient determined to have LSIL (mild dysplasia) according to the Bethesda system. FIG. 2A shows the results obtained when Alexa Fluor488 was used to cause fluorescence, and the regions outlined with a dotted line are stained regions. FIG. 2B shows the results obtained when DAB was used to cause color development. FIG. 2C shows the results obtained when Fast Red was used to cause color development. In each of FIGS. 2A to 2C, the upper row shows the result obtained regarding the HSIL specimen and the lower row shows the result obtained regarding the LSIL specimen. It was found that there was no difference in stainability among these color development methods, and all the color development methods exhibited staining behavior reflecting the reactivity with MUC1. Similarly to the case of the observation using the tissue sections, a high level expression of MUC1 also was observed in the atypical cells appearing in the liquid-based cytology (LBC) specimens. The results of causing color development using the different labels in order to avoid arbitrary operations also support that the observation has no problem.

As can be seen in FIGS. 2A to 2C, when any of the labels was used, a high level expression of MUC1 was observed by using the anti-MUC1 antibody.

### Example 3

The present example examined the stainability for specimens collected from epithelia of the uterine cervices using different types of anti-MUC1 antibodies against MUC1 as an antigen.

The specimens were collected from a patient having CIN2 (moderate dysplasia) and a patient positive for a tumor marker SCC for cervical cancer.

As in Example 1, a monoclonal antibody 1 available from Abcam (trade name: Anti-MUC1 antibody [M4H2] ab10120, available from Abcam, clone name: M4H2; epitope GVTSAPDTRPAPGSTAPPAHGVTSA) or a monoclonal antibody 2 (trade name: Novocastra™ Lyophilized Mouse Monoclonal Antibody Muc-1 Glycoprotein NCL-MUC-1, clone name: Ma695, cell line ZR75-1, available from Leica) was used as a primary antibody (anti-MUC1 antibody). Thereafter, immunostaining was performed in the same manner as in Example 1.

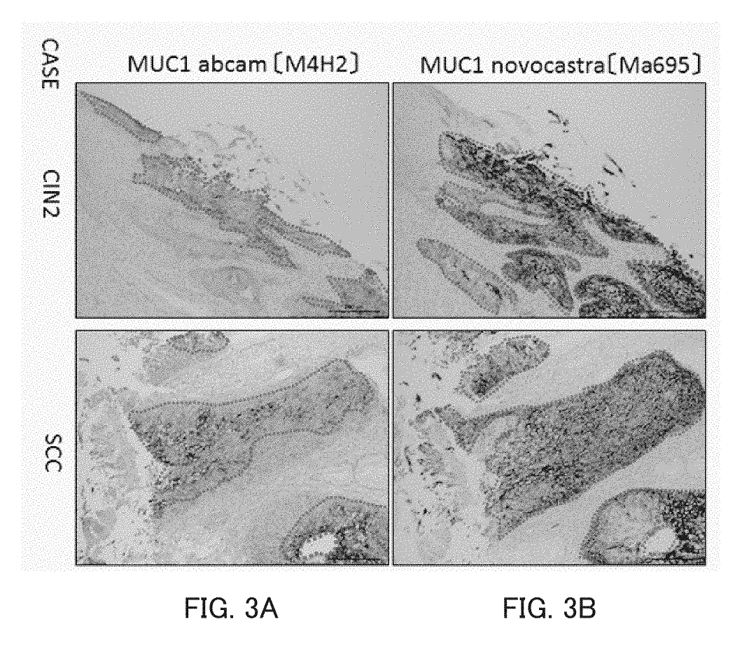

The results obtained are shown in FIGS. 3A and 3B. FIGS. 3A and 3B show the results of immunostaining using the respective anti-MUC antibodies. In FIGS. 3A and 3B, the regions outlined with a dotted line are stained regions, and the upper row shows the results obtained regarding the CIN2 specimen and the lower row shows the results obtained regarding the SCC specimen. FIG. 3A shows the results obtained when the monoclonal antibody 1 was used. FIG. 3B shows the results obtained when the monoclonal antibody 2 was used.

As shown in FIGS. 3A and 3B, the stainability of the two types of anti-MUC1 antibodies for the CIN2 and SCC specimens was examined. As a result, when either of the antibodies was used, the MUC1 expression could be observed in the CIN2 and SCC specimens. In particular, as can be seen in FIG. 3B, the monoclonal antibody 2 [Ma695] exhibited the staining behavior excellent in specificity and sensitivity.

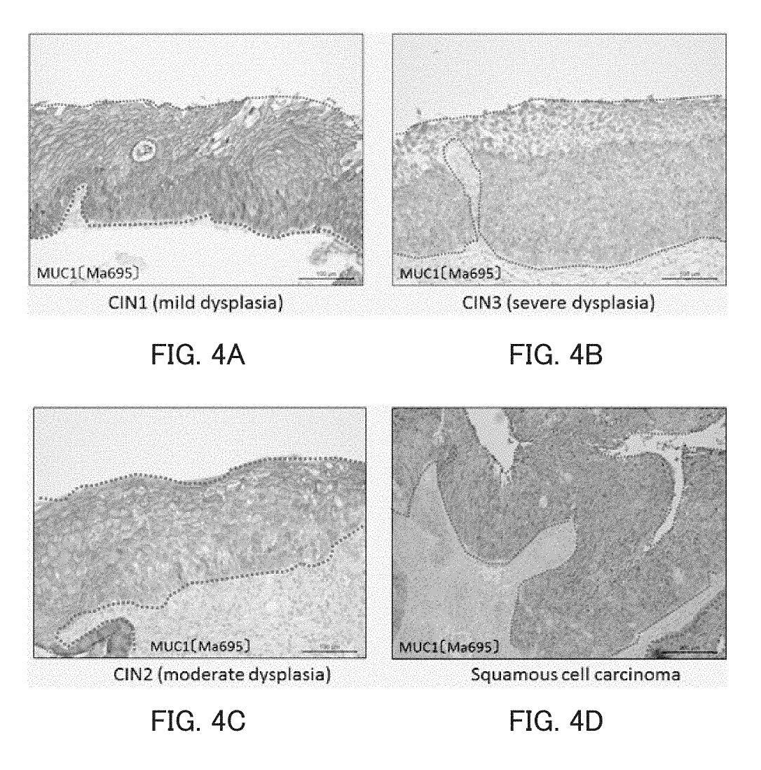

Further, regarding a CIN1 patient, a CIN2 patient, a CIN3 patient, and a SCC positive patient, immunostaining was performed in the same manner as in Example 1 using, as the anti-MUC1 antibody, the monoclonal antibody 2 [Ma695] as in the above. The results obtained are shown in FIGS. 4A to 4D. In FIGS. 4A to 4D, the regions outlined with a dotted line are stained regions. FIG. 4A shows the result obtained regarding the CIN1 specimen. FIG. 4B shows the result obtained regarding the CIN3 specimen. FIG. 4C shows the result obtained regarding the CIN2 specimen. FIG. 4D shows the result obtained regarding the SCC positive specimen. In FIGS. 4A to 4C, the bar indicates a length of 100 μm. In FIG. 4D, the bar indicates a length of 200 μm. As can be seen in FIGS. 4A to 4D, it was found that, regardless of the progression of endocervix lesion, strong stainability can be obtained generally over the full thickness from a basal layer to a cortical layer.

### Example 4

The present example examined whether, in cervical epithelial tissue, the boundary between normal squamous epithelial tissue and cervical intraepithelial lesion can be determined by localization of MUC1.

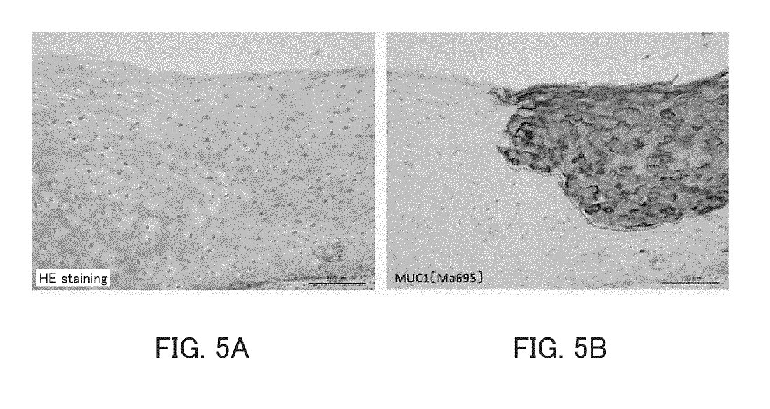

Specimens collected from CIN1 patients (15 cases) were used, and immunostaining was performed in the same manner as in Example 1 using the anti-MUC1 antibody [Ma695] as the primary antibody. The results obtained are shown in FIGS. 5A and 5B. In FIGS. 5A and 5B, the region outlined with a dotted line is a stained region, and the bar indicates a length of 100 μm. FIG. 5A shows the result of the HE staining. FIG. 5B shows the result of immunostaining performed with respect to the same specimen. As can be seen in FIGS. 5A and 5B, the immunostaining using the anti-MUC1 antibody [Ma695] clarified the boundary between the stained region and the unstained region, which revealed the localization of MUC1 expression. These results demonstrate that immunostaining using the anti-MUC1 antibody [Ma695] stains cervical intraepithelial lesions without staining normal squamous epithelial tissue, so that cervical intraepithelial lesions can be determined specifically with high sensitivity.

### Example 5

The present example examined the stainability of MUC1 in endometrial glandular cells (EC) in the uterine cervices.

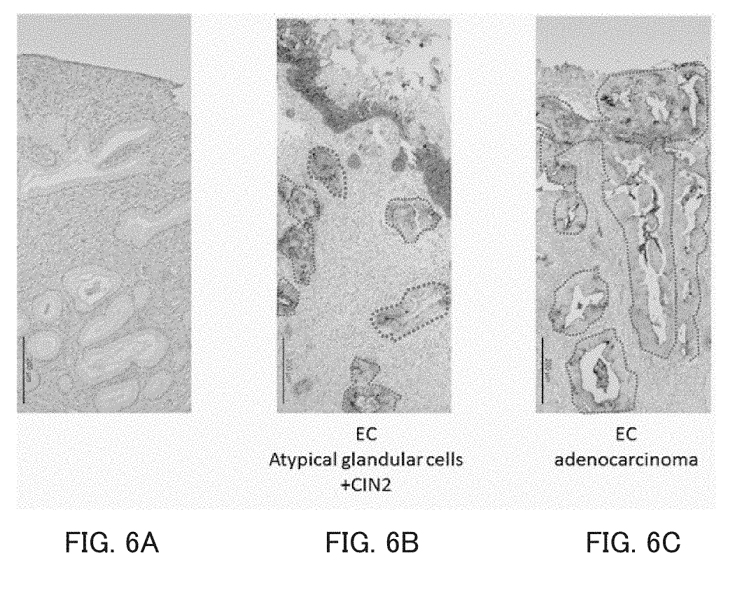

5 specimens of normal glandular cells, 5 specimens of atypical glandular cells (AGC), and 9 specimens of adenocarcinoma cells collected from patients were used, and immunostaining was performed in the same manner as in Example 1 using the anti-MUC1 antibody [Ma695] as the primary antibody. The results obtained are shown in FIGS. 6A to 6C. In FIGS. 6A to 6C, the regions outlined with a dotted line are stained regions, and the bar indicates a length of 200 μm. FIG. 6A shows the result obtained regarding the normal glandular cells. FIG. 6B shows the result obtained regarding the atypical glandular cells. FIG. 6C shows the result obtained regarding the adenocarcinoma cells. As can be seen in FIGS. 6A to 6C, according to the immunostaining using the anti-MUC1 antibody [Ma695], the atypical glandular cells and the adenocarcinoma cells were stained while the normal glandular cells were not stained, whereby the MUC1 expression could be observed.

### Example 6

Regarding specimens determined to be HSIL (high grade squamous intraepithelial lesion), ASC-US (atypical squamous cells of undetermined significance), and ASC-H (atypical squamous cells that cannot exclude HSIL) according to the Bethesda system, the MUC1 expression was examined using an anti-MUC1 antibody.

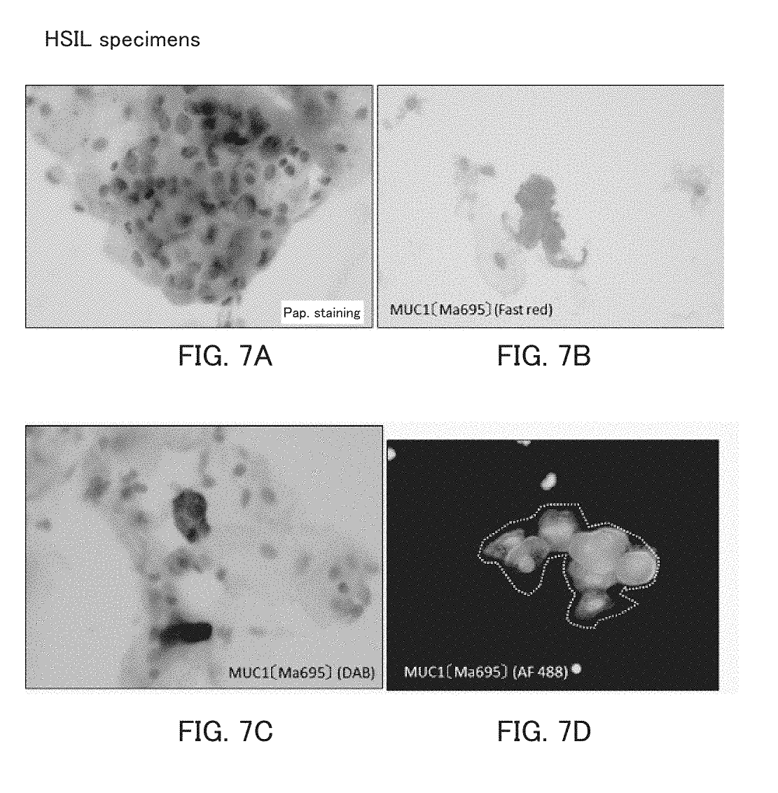

Regarding the above described specimens (19 cases), immunostaining was performed in the same manner as in Example 1 using the anti-MUC1 antibody [Ma695] as the primary antibody. The results obtained are shown in FIGS. 7A to 9D. In FIGS. 7A to 9D, the regions outlined with a dotted line are stained regions.

FIGS. 7A to 7D show the results obtained regarding the HISL specimens. FIG. 7A shows the result of Papanicolaou staining. FIG. 7B shows the result of staining MUC1 by color development using Fast red. FIG. 7C shows the result of staining MUC1 by color development using DAB. FIG. 7D shows the result of causing MUC1 to develop fluorescence using Alexa Fluor 488. As can be seen in FIG. 7A, as a result of the Papanicolaou staining, a cell mass with little disintegration was observed, and in the cell mass, swelling and size variation of nuclei were conspicuous. Also, from the color development in FIGS. 7B to 7C and the fluorescence in FIG. 7D resulting from the detection of MUC1 expression, it was presumed that the cells being examined were atypical squamous cells and atypical endometrial glandular cells in the uterine cervices.

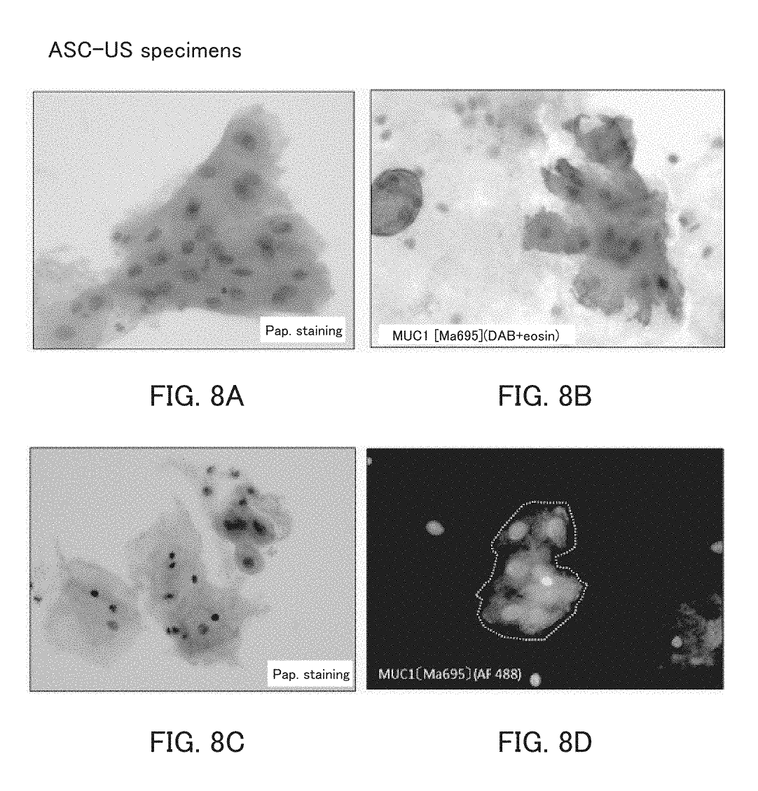

FIGS. 8A to 8D show the results obtained regarding the ASC-US specimens. FIG. 8A shows the result of Papanicolaou staining. FIG. 8B shows the result of staining MUC1 by color development using DAB and then post-staining the MUC1 with eosin. FIG. 8C shows the result of Papanicolaou staining. FIG. 8D shows the result of causing MUC1 to develop fluorescence using Alexa Fluor 488. As can be seen in FIG. 8A, as a result of the Papanicolaou staining, a cell mass with a low nucleus-cytoplasm (N/C) ratio was observed while swelling of nuclei was observed. From the color development in FIG. 8B resulting from the detection of MUC1 expression, it was presumed that the cells being examined were atypical squamous cells and atypical endometrial glandular cells in the uterine cervix. Further, as can be seen in FIG. 8C, as a result of the Papanicolaou staining, substantially round cells with mild swelling of nuclei were observed. From the fluorescence in FIG. 8D resulting from the detection of MUC1 expression, it was presumed that the cells being examined were atypical squamous cells and atypical endometrial glandular cells in the uterine cervix.

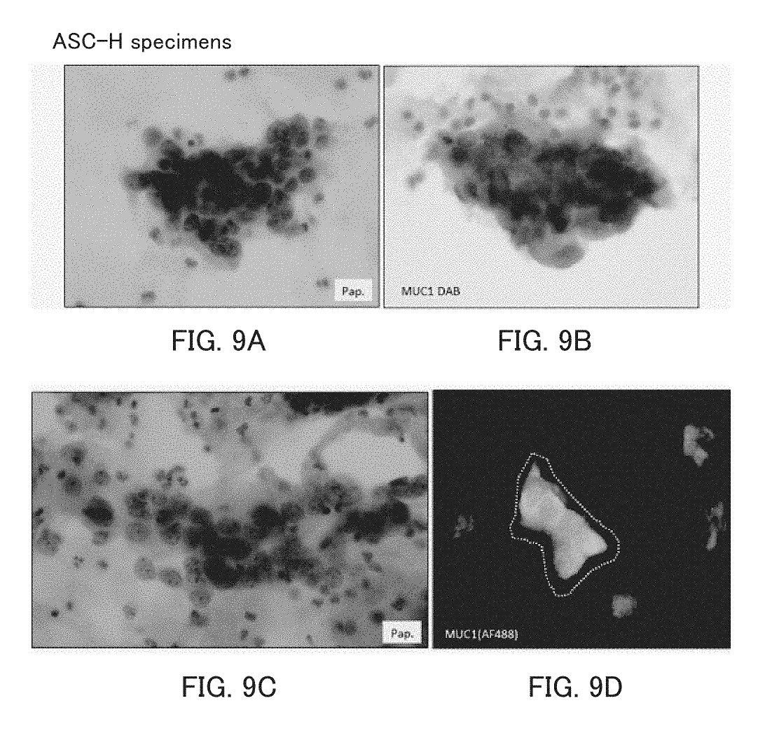

FIGS. 9A to 9D show the results obtained regarding the ASC-H specimens. FIG. 9A shows the result of Papanicolaou staining. FIG. 9B shows the result staining MUC1 by color development using DAB. FIG. 9C shows the result of Papanicolaou staining. FIG. 9D shows the result of causing MUC1 to develop fluorescence using Alexa Fluor 488. As can be seen in FIG. 9A, as a result of the Papanicolaou staining, a high density cell mass of atypical glandular cells in the uterine cervix with swelling of nuclei was observed. From the color development in FIG. 9B resulting from the detection of MUC1 expression, it was presumed that the cells being examined were atypical squamous cells and atypical endometrial glandular cells in the uterine cervix. Further, as can be seen in FIG. 9C, as a result of the Papanicolaou staining, atypical glandular ducts covered with mucus and inflammatory cells were observed. From the fluorescence in FIG. 9D resulting from the detection of MUC1 expression, it was presumed that the cells being examined were atypical squamous cells and atypical endometrial glandular cells in the uterine cervix.

As described above, the results shown in FIGS. 7A to 9D demonstrate that cell groups reactive with an anti-MUC1 antibody in cervical lesions can be determined to be atypical squamous cells and atypical endometrial glandular cells in the uterine cervix.

### Example 7

Regarding specimens determined to be negative for intraepithelial lesion or malignancy (NILM) according to the Bethesda system, the MUC1 expression was examined using an anti-MUC1 antibody.

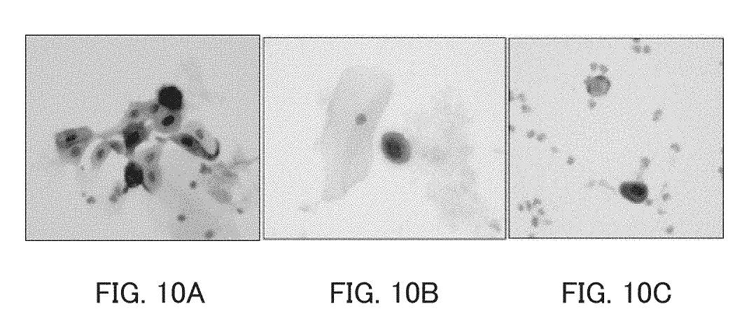

NILM specimens were prepared from the following patients (A) to (C) determined to be NILM as a result of examining the Pap. specimen slides: a patient (A) determined to be NILM on the ground that the examined cells were recognized as metaplastic cells; a patient (B) determined to be NILM on the ground that only a few small lesions were observed; and a patient (C) determined to be NILM on the ground that no lesion was observed. The MUC1 expression was examined in the same manner as in Example 1 using the anti-MUC1 antibody [Ma695] as the primary antibody. The results obtained are shown in FIGS. 10A to 10C. FIGS. 10A, 10B, and 10C show the results obtained regarding the three patients (A, B, and C), respectively. As can be seen in FIG. 10, in all the patients, staining with the anti-MUC1 antibody was observed, which revealed the MUC1 expression. The above-described respective examples verified a high correlation between cervical cancer and the MUC1 expression. Accordingly, it can be said that, even for patients determined to be NILM according to the Bethesda system, findings corresponding to ASC-US or ASC-H may be obtained by examining the MUC1 expression.

While the present invention has been described above with reference to illustrative embodiments, the present invention is by no means limited thereto. Various changes and modifications that may become apparent to those skilled in the art may be made in the configuration and specifics of the present invention without departing from the scope of the present invention.

This application claims priority from Japanese Patent Application No. 2015-200598 filed on Oct. 8, 2015. The entire disclosure of this Japanese patent application is incorporated herein by reference.

### INDUSTRIAL APPLICABILITY

As specifically described above, according to the present invention, the possibility of cervical cancer can be evaluated easily by merely detecting the presence or absence of MUC in a biological sample isolated from the uterine cervix.

1. A test method for evaluating a possibility of cervical cancer, the test method comprising the step of:
detecting expression of a MUC in a biological sample isolated from the uterine cervix.

2. The test method according to claim 1, wherein
the detection step is a step of detecting the expression of the MUC using a binding substance that binds to the MUC.

3. The test method according to claim 2, wherein
in the detection step, the expression of the MUC is detected indirectly by detecting the binding substance that has bound to the MUC.

4. The test method according to claim 2, wherein
the expression of the MUC is detected by detecting a signal generated directly or indirectly through binding of the binding substance with the MUC.

5. The test method according to claim 1, wherein
it is determined that the biological sample has a possibility of cervical cancer when the MUC is detected in the biological sample in the detection step.

6. The test method according to claim 1, wherein
the binding substance is an anti-MUC antibody.

7. The test method according to claim 1, wherein
the MUC is MUC1.

8. The test method according to claim 1, wherein
the biological sample is a cell or a tissue.

9. The test method according to claim 1, wherein
the biological sample is derived from an epithelium of the uterine cervix.

10. A test reagent for cervical cancer, the test reagent comprising:
a binding substance that binds to a MUC.

11. The test reagent according to claim 10, wherein the binding substance is an anti-MUC antibody.

12. The test reagent according to claim 10 or 11, wherein the MUC is MUC1.
